(19) United States
(12) Patent Application Publication
(10) Pub. No.: US 2012/0022047 A1
(43) Pub. Date: Jan. 26, 2012

(54) PROCESS FOR THE PURIFICATION OF ESLICARBAZEPINE ACETATE

(75) Inventors: Joseph Prabahar Koilpillai, Tirunelveli (IN); Pravin Bhalchandra Kulkarni, Kal Yan (W) (IN); Sachin Bapurao Sawant, Kolhapur (IN); Nagesh Devidasrao Limbekar, Parbhani (IN)

(73) Assignee: GLENMARK GENERICS LTD, MUMBAI (IN)

(21) Appl. No.: 13/260,914

(22) PCT Filed: Mar. 30, 2010
(86) PCT No.: PCT/IN10/00207

§ 371 Date: Sep. 28, 2011

(30) Foreign Application Priority Data
Apr. 2, 2009 (IN) 903/MUM/2009
Aug. 12, 2009 (IN) 1860/MUM/2009

(51) Int. Cl. A61K 31/55; A61P 25/08; C07D 223/28

(52) U.S. Cl. 514/217; 540/589

(57) ABSTRACT

The present invention relates to the purification and particle size of eslicarbazepine acetate. The present invention also relates to the physical characteristics of solid state eslicarbazepine acetate, and pharmaceutical compositions containing the same.

### PRIORITY

This application claims the benefit to Indian Provisional Applications 903/MUM/2009, filed on Apr. 2, 2009 and 1888/MUM/2009, filed on Aug. 12, 2009, the contents of which, are incorporated by reference herein.

### BACKGROUND OF THE INVENTION

1. Technical Field

The present invention relates to the purification of and particle size of (S)-(−)-10-(acetyloxy)-10,11-dihydro-5H-dibenz[b,f]-azepine-5-carboxamide (eslicarbazepine acetate). The present invention also relates to the physical characteristics of solid state eslicarbazepine acetate, and pharmaceutical compositions containing the same.

2. Description of the Related Art

Eslicarbazepine acetate is a novel, voltage-gated sodium channel blocker that has been studied to reduce the frequency of partial-onset seizures when used in combination with other anti-epileptic drugs. Eslicarbazepine acetate under the name ZEBINIX® in the European Union, is under review for the treatment of partial-onset seizures with or without secondary generalization in combination with other anti-epileptic drugs. Eslicarbazepine acetate is chemically known as (S)-(−)-10-(acetyloxy)-10,11-dihydro-5H-dibenz[b,f]-azepine-5-carboxamide and represented by the formula as shown below:

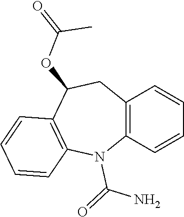

U.S. Pat. No. 5,753,646 describes dihydrodibenzo[b,f]azepines derivatives, including eslicarbazepine acetate or stereoisomer thereof, a pharmaceutical composition, a method of treatment, and a process for the preparation of eslicarbazepine acetate.

J. Med. Chem., 42, 2582-2587 (1999) discloses the preparation of eslicarbazepine of formula (I) by esterification of racemic 10,11-dihydro-10-hydroxy-5H-dibenz [b,f]azepine-5-carboxamide of formula (II) with mentyloxyacetic acid, then the separation of the resulting diastereomers and hydrolysis of the respective mentyloxyacetate which is illustrated below:

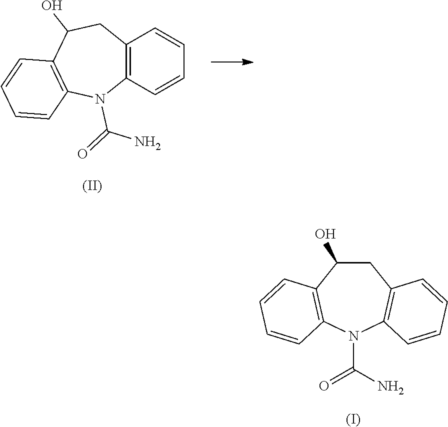

U.S. Pat. Nos. 7,119,197, 7,189,846 and 7,241,886 also disclose processes for preparation of eslicarbazepine or eslicarbazepine acetate.

PCT Patent Publication WO2006/056339 discloses a process for the preparation of (S)-(+)-10,11-dihydro-10-hydroxy-5H-dibenz[b, f]azepine-5-carboxamide (eslicarbazepine) of formula (I) from racemic 5-cyano-10,11-dihydro-10-hydroxy-5H-dibenz [b, f]azepine.

U.S. Patent Publications 2006/0142566, 2008/0221320, 2008/0139807 and PCT Patent Publications 2007/117166 and 2007/012793 also disclose processes for preparation of eslicarbazepine or eslicarbazepine acetate.

U.S. Patent publication 2007/0196488 describes a pharmaceutical composition comprising eslicarbazepine having a median particle size between 20 μm and 50 μm.

There are evolving and more rigorous requirements demanded of drug manufacturers. There are usually prevailing challenges with synthesis or manufacture in prior art, prompting a need for an improved process for the preparation of eslicarbazepine acetate, which circumvents the use of potentially hazardous chemicals, the likely formation of isomeric and other process related impurities, while ensuring a target eslicarbazepine product with optimum yield and purity.

The objective of the present invention is to provide a method for the purification of eslicarbazepine acetate in good yield and high purity.

The simple, eco-friendly, inexpensive, reproducible, robust processes, herein described for the purification of eslicarbazepine acetate are well suited on an industrial scale.

### SUMMARY OF THE INVENTION

The present invention relates to the purification and particle size of eslicarbazepine acetate.

The present invention also relates to the physical characteristics of solid state eslicarbazepine acetate, and pharmaceutical compositions containing the same.

The present invention provides crystalline particles of eslicarbazepine acetate, having a purity greater than about 99.0% as measured by high performance liquid chromatography (HPLC).

The present invention provides eslicarbazepine acetate, characterized by an X-ray Powder Diffraction (XRPD) spectrum, which is substantially in accordance with FIG. 1.

The present invention provides eslicarbazepine acetate, characterized by Differential Scanning calorimetry (DSC) endotherm, which is substantially in accordance with FIG. 2.

The present invention provides crystalline particles of eslicarbazepine acetate having a median particle size (d50) below about 20 μm.

The present invention further provides crystalline particles of eslicarbazepine acetate having a median particle size (d50) between about 5 μm to about 20 μm.

The present invention provides crystalline particles of eslicarbazepine acetate having a specific surface area of from about 0.1 m²/g to about 10 m²/g as measured by Brunauer-Emmett-Teller (B.E.T) method.

The present invention further provides crystalline particles of eslicarbazepine acetate, wherein the particles have a specific surface area from about 0.5 m²/g to about 5 m²/g as measured by Brunauer-Emmett-Teller [B.E.T] method.

The present invention provides crystalline particles of eslicarbazepine acetate, characterized by Thermogravimetric analysis (TGA) graph, which is substantially in accordance with FIG. 3.

The present invention provides crystalline particles of eslicarbazepine acetate, wherein the particles have an aggregate crystal particle shape as observed by scanning electron microscope (SEM), which is substantially in accordance with FIG. 4.

The present invention provides a process for purifying eslicarbazepine acetate comprising:

a) providing a solution of eslicarbazepine acetate in a solvent or a mixture of solvents or their aqueous mixtures and
b) precipitating the solid from the solution, and
c) recovering the eslicarbazepine acetate in substantially pure form.

The present invention provides a pharmaceutical composition comprising eslicarbazepine acetate and at least a pharmaceutically acceptable carrier.

### BRIEF DESCRIPTION OF THE DRAWINGS

FIG. 1: X-ray Powder diffraction Pattern (XRPD) of eslicarbazepine acetate prepared by Example 5.

FIG. 2: Differential Scanning calorimetry (DSC) endotherm of eslicarbazepine acetate prepared by Example 5.

FIG. 3: Thermogravimetric Analysis (TGA) graph of eslicarbazepine acetate prepared by Example 5.

FIG. 4 Scanning Electron Micrograph (SEM) of eslicarbazepine acetate crystal particles prepared by Example 5.

### DETAILED DESCRIPTION OF THE INVENTION

The present invention is directed to a method for the purification of eslicarbazepine acetate.

The phrase, “substantially pure”, as used herein, is intended to mean eslicarbazepine acetate, having a purity equal to or greater than about 98%, preferably equal to or greater than about 99% and preferably equal to or greater than about 99.5% and also having a relatively low content of organic volatile impurities.

In an embodiment, the present invention provides eslicarbazepine acetate, having purity greater than about 96.0% to about 99.9%, preferably greater than about 99.0% to about 99.8%, more preferably greater about 99.5% to about 99.8%.

In yet another embodiment, the present invention provides eslicarbazepine acetate having individual impurities lower than about 1.0%, preferably lower than about 0.5%, more preferably lower than about 0.15%.

The chemical purity of eslicarbazepine acetate was measured by high performance liquid chromatography equipped with quaternary gradient pumps, variable wavelength UV detector attached with data recorder and integrator software under the following conditions:

| | |
|---|---|
| Column: | Inertsil ODS 3V, 250 × 4.6 mm, 5μ |
| Column temperature: | 25° C. |
| Mobile Phase: | |
| Mobile Phase A: | Buffer |
| Buffer: | Adjust pH of water with 3.25 with o-phosphoric |
| | acid |
| Mobile Phase B: | Acetonitrile |

| Time (minutes) | % Mobile phase A | % Mobile phase B |
|---|---|---|
| 0.0 | 80 | 20 |
| 05 | 80 | 20 |
| 50 | 20 | 80 |
| 60 | 20 | 80 |
| 65 | 80 | 20 |
| 70 | 80 | 20 |

| | |
|---|---|
| Diluent: | Water:Acetonitrile (1:1, v/v) |
| Flow Rate: | 10. mL/minute |
| Detection: | UV 215 nm |
| Injection Volume: | 20 μL |

Dissolution and bioavailability of poorly soluble drugs may be enhanced by known practices in the art as norms of formulation to one of ordinary skill in the art. These may include the use of small particles of these drugs, exhibiting a narrow particle size distribution.

The difference in the physical properties of different solid state forms results from the orientation and intermolecular interactions of adjacent molecules or complexes in the bulk solid.

The solid state properties of eslicarbazepine acetate provides a new opportunity to improve the performance of the active pharmaceutical ingredient, providing solid state characterizations of eslicarbazepine acetate which can enhance the stability, flowability, and solubility paving a way to enhanced bioavailability and also stable pharmaceutical preparations. The solid state characterizations of a compound may also affect its behavior on compaction and its storage stability.

Thus, there is a need in the art to provide solid state characterizations of eslicarbazepine acetate. The availability of solid state characterizations of eslicarbazepine acetate would be an added advantage in the preparation of pharmaceutical formulations for the treatment of hypertension.

The present invention provides solid state characterizations of eslicarbazepine acetate

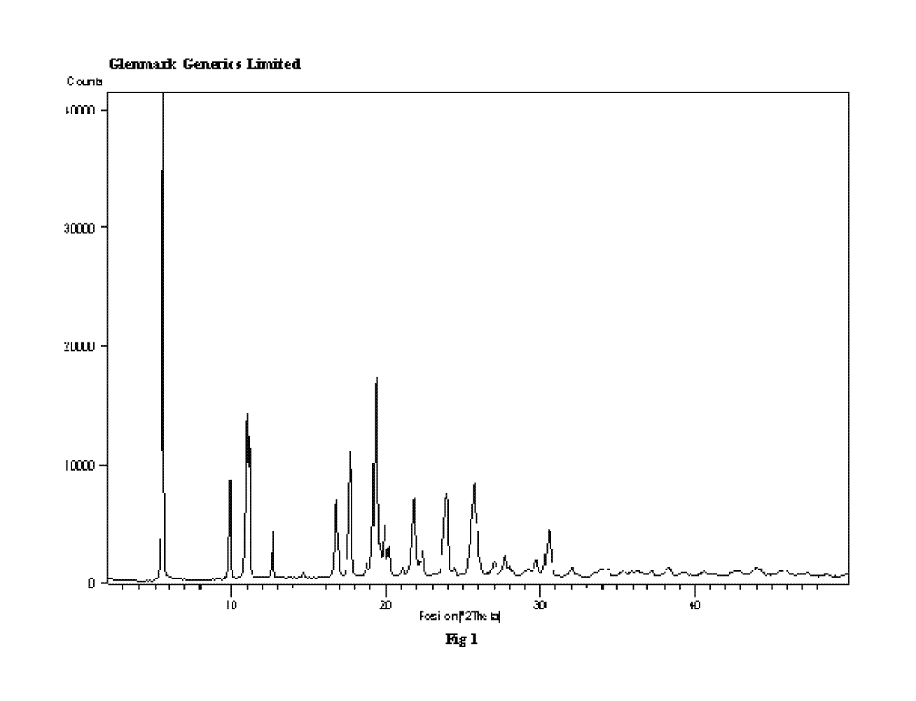

The present invention provides eslicarbazepine acetate in solid state with X-ray powder diffraction pattern, which is substantially characterized in FIG. 1, X-ray powder were performed on ARL (scanting) X-ray diffractometer model XPERT-PRO (PANalytical) scanning parameters start position [⁰2Th.] 2.01 and end position [⁰2Th.] 49.98.

Eslicarbazepine acetate is characterized by XRD peaks at 5.59, 9.92, 11.03, 11.19, 12.70, 14.73, 16.80, 17.77, 18.82, 19.39±0.2° 2theta, which substantially in accordance with FIG. 1.

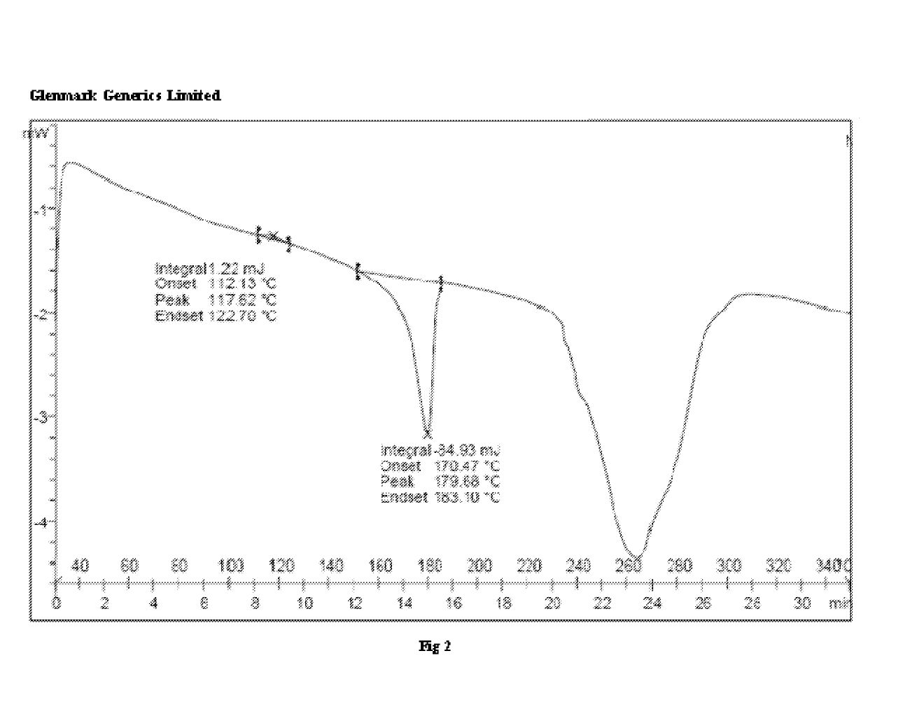

The present invention further provides eslicarbazepine acetate in solid state with a differential scanning calorimetry thermogram, which is substantially characterized in FIG. 2, is measured by a Differential Scanning calorimeter (DSC 822, Mettler Toledo) at a scan rate of 10° C. per minute with an Indium standard. Eslicarbazepine acetate exhibits an endotherm peak at about 179.68° C. Whereupon, the endotherm measured by a particular differential scanning calorimeter is dependent upon a number of factors, including the rate of heating (i.e., scan rate), the calibration standard utilized, instrument calibration, relative humidity, and upon the chemical purity of the sample being tested. Thus, an endotherm as measured by DSC on the instrument identified above may vary as much as ±1° C. or even ±2° C.

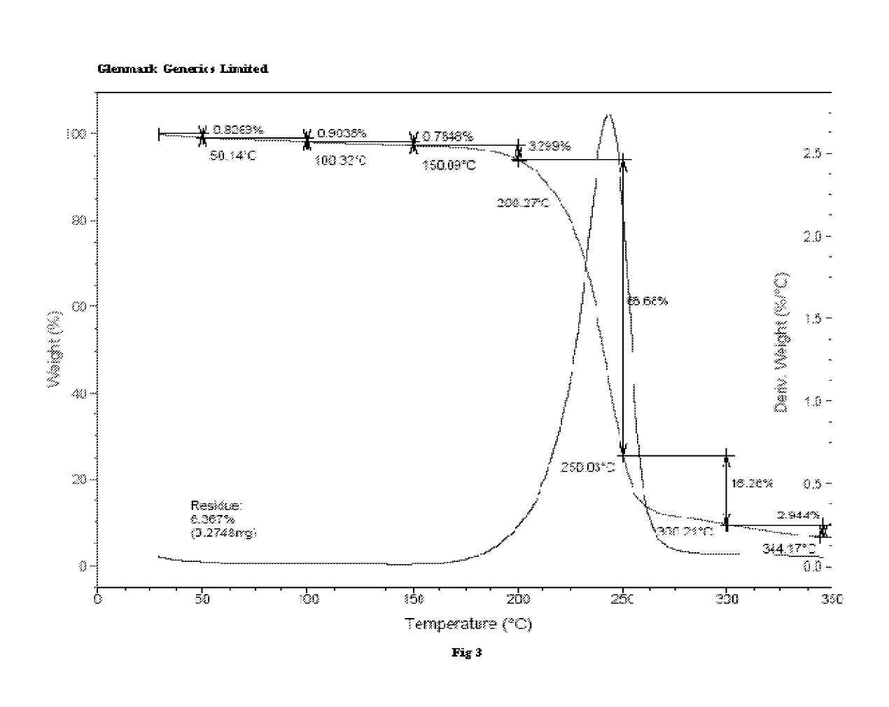

The present invention further provides eslicarbazepine acetate in solid state with a thermogravimetric analysis (TGA) scan, which is substantially characterized in FIG. 3, recorded on TGA Q500 V 20.6 in a platinum pan with a temperature rise of 10° C./min in the range 30° C. to 350° C. Moisture content of about 2.368% w/w by TGA (Thermogravimetric Analysis) which is substantially in accordance with the FIG. 3.

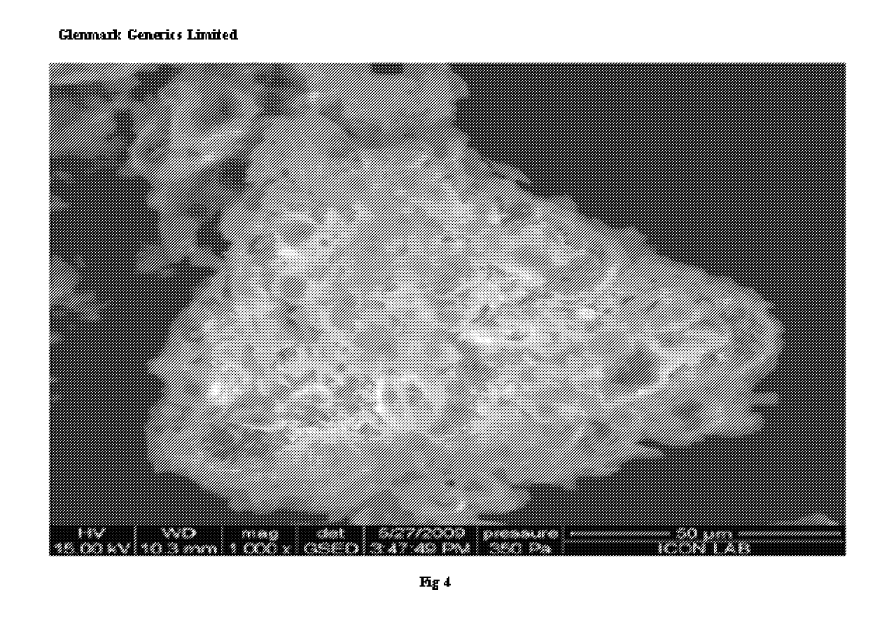

The present invention further provides eslicarbazepine acetate in crystal particles are of aggregate crystal morphology as observed by scanning electron microscope (SEM), which is substantially in accordance with FIG. 4.

Racemic (±)-10,11-dihydro-10-hydroxy-5H-dibenz[b,f]azepine-5-carboxamide of formula (II) was found to be the main metabolite of oxcarbazepine, which is synthesized by reduction of oxcarbazepine, and thus serve as a useful intermediate in the synthesis of (S)-(+)-10,11-dihydro-10-hydroxy-5H-dibenz[b,f]azepine-5-carboxamide (eslicarbazepine) of formula (I). Procedures for making oxcarbazepine are found in literature. Illustratively, it is described in U.S. Pat. No. 7,459,553 which is incorporated herein by reference, in its entirety.

Eslicarbazepine acetate can be prepared by processes described in the art. Illustratively, a process is described in U.S. Pat. No. 5,753,646, which is incorporated herein by reference in its entirety.

After completion of the reaction, the desired compounds can be obtained from the reaction mixture by conventional means known in the art. For example, the working-up of reaction mixtures, especially in order to isolate desired compounds, follows customary procedures, known to the organic chemists skilled in the norms of the art and steps, e.g. selected from the group comprising but not limited to extraction, neutralization, crystallization, chromatography, evaporation, drying, filtration, centrifugation and the like.

In yet another embodiment, the present invention provides a process for preparing eslicarbazepine acetate, comprising:

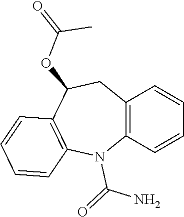

reacting the eslicarbazepine of formula (I), prepared by the processes known in the art, with an acylating agent, in the presence of an organic solvent.

The acylating agents that can be used include, but are not limited to, acetyl chloride, acetic anhydride and the like. Preferably, acetyl chloride.

The solvent that can be used include, but are not limited to methylene chloride, ethylene chloride pyridine, toluene and the like.

In yet another embodiment, the present invention provides a process for purifying eslicarbazepine acetate comprising:

a) providing a solution of eslicarbazepine acetate in a solvent or a mixture of solvents or their aqueous mixtures and
b) precipitating the solid from the solution,
and c) recovering the eslicarbazepine acetate in substantially pure form.

The solvent or mixture of solvents is selected from a C2-C5 nitrile, C2-C6 ether, a C2-C6 ester, a mixture of C2-C5 nitrile/C2-C6 ether, a mixture of C2-C6 ester/H2O, a mixture of C2-C6 ether/C3-C5 ketone, a mixture of C2-C6 ether/C2-C6 ester, a mixture of C2-C6 ether/C1-C5 alcohol, cyclic ether, hydrocarbon solvents and their halogenated derivatives, a C3-C5 carbonate, polar solvent such as dimethylformamide, dimethylsulfoxide, dimethyl acetamide and mixtures thereof, and mixtures of said organic solvents and water. Preferably acetonitrile, methyl tertiary butyl ether, methyl tertiary butyl methyl ether, tetrahydrofuran, methyl ethyl ketone, n-hexane and mixtures thereof, and mixtures of said organic solvents and water. The C2-C5 nitrile include acetonitrile, propionitrile and the like; C2-C6 ether include dimethyl ether, diethyl ether, isopropyl ether, methyl tertiary butyl ether (MTBE), methyl tertiary butyl methyl ether (MTBME); C2-C6 ester include ethyl acetate, isopropyl acetate, isobutyl acetate, t-butyl acetate and the like; C3-C5 ketone include acetone, methyl ethyl ketone, ethyl methyl ketone and the like; C1-C5 alcohol include methanol, ethanol, isopropanol, isobutanol, 2-butanol and the like; cyclic ether include tetrahydrofuran (THF), dioxane and the like; C3-C5 carbonate include dimethyl carbonate, diethyl carbonate and the like; hydrocarbon solvents and halogenated derivatives thereof may include pentane, n-hexane, heptane, cyclohexane, petroleum ether, m-, o-, or p-xylene, dichloromethane (MDC), chloroform, carbon tetrachloride, 1,2-dichloroethane and the like.

The temperature for dissolution can range from about 25° C. to about 100° C. or reflux temperatures of the solvents used, preferably at about 30° C. The time period for dissolution can be range from about 30 minutes to about 5 hours, preferably 1 hour. The solution obtained is optionally filtered through celite or diatamous earth to separate the extraneous matter present or formed in the solution by using conventional filtration technique known in the art.

The precipitation of solid in b) above is achieved but not limited to evaporation, cooling, drying, by adding antisolvent and the like. Preferably by adding antisolvent.

The temperature range for precipitation of solid can be from about −10° C. to about 30° C., preferably about 30° C.

The time period for complete precipitation of solid can range from about 30 minutes to about 5 hours, preferably 1 hour.

The obtained esclicarbazepine acetate can be dried can be from about 25° C. to about 75° C., preferably at 50° C. and at reduced pressure of about e.g. 5 to 20 mbar, for a period of about 1 to about 10 hours. Preferably 1 hour.

The solid state characterizations of a compound may also affect its behavior on compaction and its storage stability. The solid state properties of eslicarbazepine acetate provides a new opportunity to improve the performance of the active pharmaceutical ingredient (API). Solid state characterizations of eslicarbazepine acetate may lead to the enhancement of the stability, flowability, and solubility of the corresponding API paving a way to enhanced bioavailability and also stable pharmaceutical preparations. The availability of solid state characterizations of eslicarbazepine acetate would be an added advantage in the preparation of pharmaceutical formulations for the treatment of hypertension.

According to another aspect, the present invention provides crystalline particles of eslicarbazepine acetate having a specific surface area of from about 0.1 m2/g to about 10 m2/g.

The present invention provides eslicarbazepine acetate particles has a specific surface area of from about 0.7 to 3.5 m2/g, and more preferably of from about 0.5 to about 2.0 m2/g.

The present invention provides crystalline particles of eslicarbazepine acetate having a median particle size (d50) below about 20 μm.

The present invention further provides crystalline particles of eslicarbazepine acetate having a median particle size (d50) between about 5 μm to about 20 μm.

As used herein, the term “μm” refers to “micrometer” which is 1×10˜6 meter.

As used herein, “crystalline particles” means any combination of single crystals, aggregates and agglomerates.

As used herein, the phrase, “particle size distribution (PSD)” means the cumulative volume size distribution of equivalent spherical diameters as determined by laser diffraction at 1 bar dispersive pressure in a Sympatec Helos equipment.

d10, as used herein is defined as the particle size at which the cumulative percentage undersize is 10 (i.e. the bottom 10% of particles are less than or equal to the stated size). d50 means the median particle size and d90 is defined as the particle size at which the cumulative percentage undersize is 90 (i.e. the bottom 90% of particles are less than or equal to the stated size).

Specific surface area is defined in units of square meters per gram (m2/g). It is usually measured by nitrogen absorption analysis. In this analysis, nitrogen is absorbed on the surface of the substance. The amount of the absorbed nitrogen (as measured during the absorption or the subsequent desorption process) is related to the surface area via a formula known as the Brunauer Emmet Teller (B.E.T.) formula.

The lack of solubility of eslicarbazepine acetate in aqueous medium poses a challenge, since the bioavailability of a water insoluble active ingredient, like eslicarbazepine acetate, is usually poor. Thus there is a need in the art to prepare active pharmaceutical ingredients, such as eslicarbazepine acetate with a high surface area to obtain formulations with greater bioavailability, and to compensate for any loss of surface area before formulation.

As eslicarbazepine acetate is pH dependent and poorly soluble in aqueous medium, it is essential to attain an eslicarbazepine acetate that has reduced particle size distribution and larger surface area, which subsequently leads to better solubility and bioavailability in aqueous medium. In view of the foregoing, there is a need in the medical arts for eslicarbazepine acetate with defined particle size distribution and surface area which are interlinked and has effect on the solubility and bioavailability.

The present invention provides eslicarbazepine acetate having desirable particle size distribution and specific surface area suitable for enhanced bioavailability and solubility in aqueous medium.

The present invention provides crystal particles of eslicarbazepine acetate obtained by the processes herein described having the following characteristics:
Particle size distribution:
d 10: 5.421 μm
d 50: 19.780 μm.
d 90: 168.686 μm
Specific surface area of about 1.37 m²/g, as measured by Brunauer-Emmett-Teller (B.E.T.)

The particle size of eslicarbazepine acetate was measured under following conditions.

| Instrument | Malvern Mastersizer |
|---|---|
| Sample Handling Unit | Hydro2000S (A) |
| Sample Preparation | Weigh accurately about 200-300 mg of |
| | well mixed sample in a beaker. Add |
| | 5-10 drops of dispersant. Make a |
| | uniform paste. Add 25 ml dispartant and |
| | stir to mix well. Disperse the sample in |
| | the dispersing media. |
| Material R.I. | 1.65 |
| Material Absorption | 0.001 |
| Dispersant Name | Liquid paraffin |
| Dispersant R.I. | 1.468 |
| Model | General purpose |
| Sensitivity | Normal |
| Particle Shape | Irregular |
| Measurement Time | 12.0 secs |
| Background Time | 12.0 secs |
| Obscuration Range | 10-20% |
| Stirrer Speed | 2500 rpm |
| Ultrasonic | 60 sec, premeasurment |
| Tip displacement (sonication) | 40% |

In accordance with the invention, the size distribution of eslicarbazepine acetate particles is determined by laser diffraction. In the present invention, the method in the determination of the size of eslicarbazepine acetate particles employed a Malvern Mastersizer laser diffraction instrument. Samples of the eslicarbazepine acetate were suspended in hexane containing a surfactant, 1% Tween80®. The suspensions were mixed and then sonicated for 120 seconds to thoroughly disperse the eslicarbazepine acetate particles. The dispersion was then circulated in the flow cell of the Malvern Mastersizer for two minutes before particle size measurements were taken.

Eslicarbazepine acetate of defined particle size may be produced by precipitation from appropriate solvents. Particle size may be adjusted by customary methods known in the art, which include cooling, pH adjustment, pouring a concentrated solution into an anti-solvent and/or by co-precipitation so as to obtain a precipitate with the appropriate particle size distribution

Eslicarbazepine acetate of defined particle size may be produced by methods known in the art for particle size reduction starting with crystals, powder aggregates and coarse powder of either crystalline or amorphous eslicarbazepine acetate. The principal operations of conventional size reduction are milling of a feedstock material and sorting of the milled material by size.

In accordance with the invention, the powder composition comprises eslicarbazepine acetate of defined particle size and optionally one or more other substances, such as pharmaceutical excipients. The powder composition of this invention may be formulated into a variety of solid and liquid dosage forms for administration to humans and animals. The dosage forms include those suitable for enteral (oral, sublingual, buccal, rectal) administration.

In yet another embodiment, eslicarbazepine acetate, obtained by the processes described above, has residual organic solvents or organic volatile impurities which fall at less than the amount recommended for pharmaceutical products, as set forth for example in ICH guidelines and U.S. pharmacopoeia; i.e., less than about 800 ppm of dichloromethane, less than about 200 ppm of acetone and methanol, ethanol and isopropyl alcohol below the detection limit.

While the present invention has been described in terms of its specific embodiments, certain modifications and equivalents will be apparent to those skilled in the art and are intended to be included within the scope of the present invention.

### EXAMPLES

### Example 1

### Preparation of racemic 10,11-Dihydro-10-hydroxy-5H-dibenz [b,f]azepine-5-carboxamide

Oxcarbazepine (50 gm, 0.20 mol) is suspended in a mixture of water (116 mL) and ethanol (203 mL). Sodium borohydride (5.81 gm, 0.15 mol) is added to this suspension in three equal portions over 15 min at about 25-30° C. The temperature of reaction mixture is raised to about 40-45° C. and continued stirring at about 40-45° C. for about 3 hours. After completion of the reaction, reaction mixture is cooled to about 10-15° C. and acetone (43.5 mL) is added at about 10-15° C. The reaction mixture is concentrated at about 40-45° C. under reduced pressure. The residue is triturated with water (125 mL) at room temperature to obtain the product as solid. The product is filtered, washed with water (25 mL) and dried at about 40-45° C. under reduced pressure to get racemic 10,11-Dihydro-10-hydroxy-5H-dibenz[b,f]azepine-5-carboxamide (46.75 gm).

### Example 2

### Preparation of S-(+)-10,11-Dihydro-10-hydroxy-5H-dibenz [b,f]azepine-5-carboxamide

L-(+)-Tartaric acid (20 gm, 0.13 mol) is stirred with acetic anhydride (51.49 gm, 0.5 mol) and a catalytic quantity of sulphuric acid (96%) at about 25-30° C. The reaction is exothermic and the temperature raise to about 60-65° C. Thereafter, the reaction mixture is heated to reflux and stirred at reflux temperature for 10 min. The reaction mass is concentrated at about 65-70° C. under reduced pressure and the remaining residue mass is co-evaporated with toluene (26 mL).

The residue is dissolved in methylene chloride (277 ml) and racemic 10,11-Dihydro-10-hydroxy-5H-dibenz[b,f]azepine-5-carboxamide (28.2 gm, 0.11 mol), pyridine (9.57 gm, 0.12 mol) and 4-dimethylaminopyridine (0.54 gm, 0.004 mol) are added to the solution. Thereafter, the reaction mixture is stirred at about 25-30° C. for forty minutes and then water (197 mL) is added. The reaction mass is stirred at about 15-20° C. for about 12 hours. The precipitated solid is filtered, washed with water (2×28 mL) and dried at 40-45° C. under reduced pressure to afford the intermediate, diacetyl tartarate half-ester (26.1 gm).

Diacetyl tartarate half ester (26 gm, 0.055 mol) is suspended in methanol (152 mL) and aqueous sodium hydroxide solution (3N, 75 ml, 0.22 mol) is added to this suspension at about 25-30° C. This reaction mixture is stirred at about 25-30° C. for about 30 min. Thereafter, the precipitated sodium bitartarate was filtered and washed with methanol (24 mL). The filtrate is concentrated at about 40-45° C. under reduced pressure and water (226 ml) is added to the residue. The resulting solution is kept at about 15-20° C. for about 16 hours. The isolated crystalline product is filtered, washed with water (2×30 mL) and dried at about 45-50° C. under reduced pressure to afford a white solid (10.8 gm). The crude product is dissolved in hot ethanol (48 mL) and left to stand at about 0-5° C. for about 16 hours. The crystalline product is filtered, washed with cold ethanol (14 mL) and dried at about 45-50° C. under reduced pressure to give S-(+)-10,11-Dihydro-10-hydroxy-5H-dibenz[b,f]azepine-5-carboxamide (6.0 gm).

### Example 3

### Preparation of S-(−)-10-Acetoxy-10,11-dihydro-5H-dibenz[b,f]azepine-5-carboxamide

S-(+)-10,11-Dihydro-10-hydroxy-5H-dibenz[b,f]azepine-5-carboxamide (3 gm, 0.01 mol), Pyridine (0.98 gm, 0.012 mol), 4-(dimethyl-amino)pyridine (0.15 gm, 0.0012 mol), and acetyl chloride (1.0 gm, 0.012 mol) in dichloromethane (30 mL) are stirred at about room temperature for about 1 hour. After completion of the reaction, reaction mixture is washed with 5% w/w aqueous hydrochloric acid solution (2×7.5 mL) followed by saturated sodium chloride solution (7.5 mL). Finally organic layer is concentrated at about 40-45° C. under reduced pressure to obtain the crude product. Crude product is crystallized from acetone (10 mL) to furnish S-(−)-10-Acetoxy-10,11-dihydro-5H-dibenz[b,f]azepine-5-carboxamide (3.13 gm).

### Example 4

### Preparation of S-(−)-10-Acetoxy-10,11-dihydro-5H-dibenz[b, f]azepine-5-carboxamide

S-(+)-10,11-Dihydro-10-hydroxy-5H-dibenz[b,f]azepine-5-carboxamide (35 gm, 0.13 mol), pyridine (12.85 gm, 0.1 mol), 4-(dimethylamino)pyridine (1.75 gm, 0.014 mol), and acetyl chloride (12.75 gm, 0.15 mol) in dichloromethane (350 ml) are stirred at room temperature for 1 hour. After completion of the reaction, the reaction mass is washed with 5% w/w aqueous hydrochloric acid solution (2×50 ml) and followed by washing with saturated aqueous sodium chloride solution (25 ml). The organic layer was concentrated at about 35-40° C. under reduced pressure to obtain crude product. This crude product is crystallized from acetone (100 ml) and dried at about 45-50° C. under vacuum to obtain S-(−)-10-Acetoxy-10,11-dihydro-5H-dibenz(b,f)azepine-5-carboxamide (30.2 gm). 1H NMR (CDCl₃): 7.6-7.15 (m, 8H, Ar—H), 6.4, 6.0 (2× bs, 1H, C10-H), 5.0 (bs, 2H, NH2) 3.6, 3.1, 2×m, 2H, C11-H), 2.1 (s, 3H, —CH3).

### Example -5

### Purification of Eslicarbazepine Acetate Using Acetonitrile and Methyl Tertiary Butyl Ether

5 gm of eslicarbazepine acetate and 25 ml of acetonitrile in a clean and dry 4 neck round bottom flask (RBF) followed by heating to about 60° C. The resultant suspension was stirred for about 10 to 15 minutes followed by cooling to about 25 to 30° C. 15 ml of methyl tertiary butyl ether was added under stifling over about 10 to 15 minutes. The resultant suspension was stirred for about 10-15 minutes. The solid separated was filtered and the solid was washed with 10 ml of methyl tertiary butyl ether. The solid obtained was dried at about 45-50° C. under vacuum for about 1 hour to afford the title compound. [HPLC Purity: 99.9%]

### Example 6

### Purification of Eslicarbazepine Acetate Using Tetrahydrofuran and N-Hexane

5 gm of eslicarbazepine acetate and 35 ml of tetrahydrofuran in a clean and dry 4 neck RBF followed by heating to about 60° C. The resultant suspension was stirred for about 10 to 15 minutes followed by cooling to about 25 to 30° C. 15 ml of n-hexane was added under stirring over about 10 to 15 minutes. The resultant suspension was stirred for about 10-15 minutes. The solid separated was filtered and the solid was washed with 10 ml of n-hexane. The solid obtained was dried at about 45-50° C. under vacuum for about 1 hour to afford the title compound.

### Example 7

### Purification Of Eslicarbazepine Acetate Using Tetrahydrofuran and Methyl Tertiary Butyl Methyl Ether

5 gm of eslicarbazepine acetate and 35 ml of tetrahydrofuran in a clean and dry 4 neck RBF followed by heating to about 60° C. The resultant suspension was stirred for about 10 to 15 minutes followed by cooling to about 25 to 30° C. 15 ml of methyl tertiary butyl ether was added under stifling over about 10 to 15 minutes. The resultant suspension was stirred for about 10-15 minutes. The solid separated was filtered and the solid was washed with 10 ml of methyl tertiary butyl ether. The solid obtained was dried at 45-50° C. under vacuum for about 1 hour to afford the title compound.

### Example 8

### Purification of Eslicarbazepine Acetate Using Tetrahydrofuran, Methyl Ethyl Ketone and N-Hexane

5 gm of eslicarbazepine acetate, 35 ml of tetrahydrofuran and 15 ml of methyl ethyl ketone in a clean and dry 4 neck RBF followed by heating to about 60° C. The resultant suspension was stirred for about 10 to 15 minutes followed by cooling to about 25 to 30° C. 15 ml of n-hexane was added under stirring over about 10 to 15 minutes. The resultant suspension was stirred for about 10-15 minutes. The solid separated was filtered and the solid was washed with 10 ml of n-hexane. The solid obtained was dried at 45-50° C. under vacuum for about 1 hour to afford the title compound.

1. Crystalline particles of eslicarbazepine acetate having a median particle size between 5 μm to 20 μm.

2. Crystalline particles of eslicarbazepine acetate having a specific surface area of from about 0.1 m²/g to about 10 m²/g as measured by Brunauer-Emmett-Teller (B.E.T.) method.

3. The crystalline eslicarbazepine acetate of claim 2, further having a specific surface area from about 0.5 m²/g to about 5 m²/g as measured by Brunauer-Emmett-Teller [B.E.T] method.

4. A process for purifying eslicarbazepine acetate comprising:
a) providing a solution of eslicarbazepine acetate in a solvent or a mixture of solvents or their aqueous mixtures and
b) precipitating the solid from the solution, and
c) recovering the eslicarbazepine acetate in substantially pure form.

5. The process of claim 4, wherein the solvent is selected from acetonitrile, methyl tertiary butyl ether, methyl tertiary butyl methyl ether, tetrahydrofuran, methyl ethyl ketone, n-hexane and mixtures thereof, and mixtures of said organic solvents and water.

6. Eslicarbazepine acetate of claim 4, having purity greater than about 99.0% as measured by high performance liquid chromatography.

7. A pharmaceutical composition comprising a therapeutically effective amount of eslicarbazepine acetate of claim 1, having purity greater than about 99.0% as measured by high performance liquid chromatography and at least a pharmaceutically acceptable carrier.
